(19) United States
(12) Patent Application Publication
(10) Pub. No.: US 2011/0160567 A1
(43) Pub. Date: Jun. 30, 2011

(54) FUNCTIONAL MRI CARDIAC OPTIMIZATION

(76) Inventors: Jeffrey E. Stahmann, Ramsey, MN (US); Scott R. Stubbs, Maple Grove, MN (US)

(21) Appl. No.: 12/974,791

(22) Filed: Dec. 21, 2010

Related U.S. Application Data
(60) Provisional application No. 61/335,152, filed on Dec. 31, 2009.
(60) Provisional application No. 61/335,068, filed on Dec. 31, 2009.

(51) Int. Cl. A61B 5/055

(52) U.S. Cl. 600/411

(57) ABSTRACT

An implantable or other ambulatory device, such as a pacer, defibrillator, or other cardiac function management device, can use imaging information, such as one or more of cardiac functional magnetic resonance imaging (fMRI) information or cardiac magnetic resonance imaging (MRI) information, such as for helping optimize one or more parameters of the implantable or other ambulatory device.

This patent application claims the benefit of priority, under 35 U.S.C. Section 119(e), to Stahmann et al., U.S. Provisional Patent Application Ser. No. 61/335,152, entitled “FUNCTIONAL MRI CARDIAC OPTIMIZATION,” filed on Dec. 31, 2009 (Attorney Docket No. 00279.I11PRV), which is incorporated herein by reference in its entirety, and also claims the benefit of priority to Bocek, U.S. Provisional Patent Application Ser. No. 61/335,068, entitled “MRI CARDIAC OPTIMIZATION,” filed on Dec. 31, 2009 (Attorney Docket No. 00279.I09PRV), which is incorporated herein by reference in its entirety.

### BACKGROUND

Implantable medical devices (IMDs) or other ambulatory medical devices can perform a variety of diagnostic or therapeutic functions. For example, an IMD can include one or more cardiac function management features, such as to monitor the heart or to provide electrical stimulation to a heart or to the nervous system, such as to diagnose or treat a subject, such as one or more electrical or mechanical abnormalities of the heart. Examples of IMDs can include pacers, automatic implantable cardioverter-defibrillators (ICDs), or cardiac resynchronization therapy (CRT) devices, among others.

Nuclear magnetic resonance imaging (MRI) is a medical imaging technique that can be used to visualize internal structure of the body. MRI is an increasingly common diagnostic tool, but can pose risks to a person with an IMD, such as a patient undergoing an MRI scan or a person nearby MRI equipment, or to people having a conductive implant.

In a MR field, an item, such as an IMD, can be referred to as “MR Safe” if the item poses no known hazard in all MRI environments. In an example, MR Safe items can include non-conducting, non-metallic, non-magnetic items, such as a glass, porcelain, a non-conductive polymer, etc. An item can be referred to as “MR Conditional” in the MR field if the item has been demonstrated to pose no known hazards in a specified MRI environment with specified conditions of use (e.g., static magnetic field strength, spatial gradient, time-varying magnetic fields, RF fields, etc.). In certain examples, MR Conditional items can be labeled with testing results sufficient to characterize item behavior in a specified MRI environment. Testing can include, among other things, magnetically induced displacement or torque, heating, induced current or voltage, or one or more other factors. An item known to pose hazards in all MRI environments, such as a ferromagnetic scissors, can be referred to as “MR Unsafe.”

### OVERVIEW

An ambulatory or implantable device, such as a pacer, defibrillator, or other cardiac rhythm management device, can imaging information, such as one or more of cardiac functional magnetic resonance imaging (fMRI) information or cardiac magnetic resonance imaging (MRI), such as for helping optimize one or more parameters of the ambulatory or implantable device.

Example 1 includes subject matter that can include an apparatus comprising: a processor circuit, configured to provide at least one cardiac function management device parameter, of an ambulatory medical device, having a value established at least in part using cardiac imaging information obtained from a magnetic resonance imaging (MRI) device.

In Example 2, the subject matter of Example 1 optionally comprises: a port, configured to be communicatively coupled to the MRI device and to the processor circuit; wherein the processor circuit is configured to establish the value of the at least one cardiac function management device parameter at least in part using the cardiac imaging information obtained from the MRI device; and wherein the cardiac imaging information obtained from the MRI device is obtained using the port.

In Example 3, the subject matter of one or any combination of Examples 1-2 optionally comprises a therapy circuit, configured to be communicatively coupled to the processor circuit and to provide a therapy to a subject; wherein the cardiac function management device parameter comprises a cardiac function management therapy control parameter configured to control at least one of a therapy timing, a therapy energy level, or a therapy location; and wherein the processor circuit is configured to control operation of the therapy circuit using the at least one cardiac function management therapy control parameter.

In Example 4, the subject matter of one or any combination of Examples 1-3 can optionally be configured such that the at least one cardiac function management device parameter has a value established using information obtained from an imaging apparatus including at least one of an echocardiogram, a computed tomography (CT) scan, a positron emission tomography (PET) scan, or an X-ray image.

In Example 5, the subject matter of one or any combination of Examples 1-4 can optionally be configured such that the processor circuit comprises a mode configured to: establish operative functionality of the ambulatory medical device that is compatible for use during an MRI procedure; and allow cardiac signal sensing during the MRI procedure.

In Example 6, the subject matter of one or any combination of Examples 1-5 can optionally be configured such that the processor circuit comprises a mode configured to vary a value of the at least one cardiac function management device parameter when a subject associated with the ambulatory medical device is undergoing an imaging procedure.

In Example 7, the subject matter of one or any combination of Examples 1-6 can optionally be configured such that the at least one cardiac function management device parameter has a value established using cardiac imaging information obtained from the MRI device including information about a septal wall motion.

In Example 8, the subject matter of one or any combination of Examples 1-7 can optionally be configured such that the establishing the value of the at least one cardiac function management device parameter comprises establishing the at least one cardiac function management device parameter associated with a decreased or minimum amount of septal wall motion indicated by the cardiac imaging information obtained from the MRI device.

In Example 9, the subject matter of one or any combination of Examples 1-8 can optionally be configured such that the at least one cardiac function management device parameter of the ambulatory medical device is configured to provide a correlation between a measurement of the ambulatory medical device to a measurement obtained using an imaging device.

Example 10 can include, or can optionally be combined with the subject matter of one or any combination of Examples 1-9 to include subject matter (such as a method, a means for performing acts, or a device-readable medium including instructions that, when performed by the device, cause the device to perform acts), comprising: establishing a value of at least one cardiac function management device parameter, of an ambulatory medical device, at least in part using cardiac imaging information obtained from an MRI device.

In Example 11, the subject matter of one or any combination of Examples 1-10 can optionally comprise instructions to provide a therapy to a subject using the at least one cardiac function management device parameter, and wherein the at least one cardiac function management device parameter comprises a cardiac function management therapy control parameter configured to control at least one of a therapy timing, a therapy energy level, or a therapy location.

In Example 12, the subject matter of one or any combination of Examples 1-11 can optionally comprise instructions such that establishing the value of the at least one cardiac function management device parameter comprises using information obtained from an imaging apparatus including at least one of an echocardiogram, a computed tomography (CT) scan, a positron emission tomography (PET) scan, or an X-ray image.

In Example 13, the subject matter of one or any combination of Examples 1-12 can optionally comprise instructions to establish operative functionality of the ambulatory medical device that is compatible for use during an MRI procedure and that allows cardiac signal sensing during the MRI procedure.

In Example 14, the subject matter of one or any combination of Examples 1-13 can optionally comprise instructions to vary the value of the at least one cardiac function management device parameter when a subject associated with the ambulatory medical device is undergoing an imaging procedure.

In Example 15, the subject matter of one or any combination of Examples 1-14 can optionally comprise instructions such that the establishing the value of the at least one cardiac function management device parameter using the cardiac imaging information obtained from the MRI device comprises using information about a septal wall motion.

In Example 16, the subject matter of one or any combination of Examples 1-15 can optionally comprise instructions such that the establishing the value of the at least one cardiac function management device parameter comprises establishing the value of the at least one cardiac function management device parameter associated with a decreased or minimum amount of septal wall motion indicated by the cardiac imaging information obtained from the MRI device.

In Example 17, the subject matter of one or any combination of Examples 1-16 can optionally comprise instructions such that the at least one cardiac function management device parameter of the ambulatory medical device is configured to provide a correlation between a measurement of the ambulatory medical device to a measurement obtained using an imaging device.

Example 18 can include, or can optionally be combined with the subject matter of one or any combination of Examples 1-17 to include subject matter (such as a method, a means for performing acts, or a device-readable medium including instructions that, when performed by the device, cause the device to perform acts) comprising: establishing, using a processor circuit, a value of at least one cardiac function management device parameter, of an ambulatory medical device, at least in part using cardiac imaging information obtained from an MRI device.

In Example 19, the subject matter of one or any combination of Examples 1-18 can optionally be performed such that the establishing the value of the at least one cardiac function management device parameter comprises using information obtained from an imaging apparatus including at least one of an echocardiogram, a computed tomography (CT) imaging information, a positron emission tomography (PET) imaging information, or an X-ray imaging information.

In Example 20, the subject matter of one or any combination of Examples 1-19 can optionally be performed such that the establishing the value of the at least one cardiac function management device parameter using cardiac imaging information obtained from the MRI device comprises using information about a septal wall motion.

In Example 21, the subject matter of one or any combination of Examples 1-20 can optionally be performed such that the establishing the value of the at least one cardiac function management device parameter comprises establishing the value of the at least one cardiac function management device parameter associated with a decreased or minimum amount of septal wall motion indicated by the cardiac imaging information obtained from the MRI device.

In Example 22, the subject matter of one or any combination of Examples 1-21 can optionally comprise varying the value of the at least one cardiac function management device parameter when a subject associated with the ambulatory medical device is undergoing an imaging procedure.

In Example 23, the subject matter of one or any combination of Examples 1-22 can optionally comprise establishing the at least one cardiac function management device parameter of the ambulatory medical device to provide a correlation between a measurement of the ambulatory medical device to a measurement obtained using an imaging device.

Example 24 can include, or can optionally be combined with the subject matter of one or any combination of Examples 1-23 to include subject matter (such as a method, a means for performing acts, or a device-readable medium including instructions that, when performed by the device, cause the device to perform acts), comprising: a processor circuit, configured to provide at least one cardiac function management device parameter, of an ambulatory medical device, having a value established at least in part using cardiac functional imaging information obtained from a functional magnetic resonance imaging (fMRI) device.

In Example 25, the subject matter of one or any combination of Examples 1-24 can optionally comprise a port, configured to be communicatively coupled to the fMRI device and to the processor circuit; wherein the processor circuit is configured to establish the value of the at least one cardiac function management device parameter at least in part using the cardiac functional imaging information obtained from the fMRI device; and wherein the cardiac functional imaging information obtained from the fMRI device is obtained using the port.

In Example 26, the subject matter of one or any combination of Examples 1-25 can optionally comprise a therapy circuit, configured to be communicatively coupled to the processor circuit and to provide a therapy to a subject; wherein the cardiac function management device parameter comprises a cardiac function management therapy control parameter configured to control at least one of a therapy timing, a therapy energy level, or a therapy location; and wherein the processor circuit is configured to control operation of the therapy circuit using the at least one cardiac function management therapy control parameter.

In Example 27, the subject matter of one or any combination of Examples 1-26 can optionally be configured such that the at least one cardiac function management device parameter has a value established using information obtained from an imaging apparatus including at least one of an echocardiogram, a computed tomography (CT) scan, a positron emission tomography (PET) scan, magnetic resonance imaging (MRI) scan, or an X-ray image.

In Example 28, the subject matter of one or any combination of Examples 1-27 can optionally be configured such that the processor circuit comprises a mode configured to: establish operative functionality of the ambulatory medical device that is compatible for use during an MRI procedure; and allow cardiac signal sensing during the MRI procedure.

In Example 29, the subject matter of one or any combination of Examples 1-28 can optionally be configured such that the processor circuit comprises a mode configured to vary a value of the at least one cardiac function management device parameter when a subject associated with the ambulatory medical device is undergoing an imaging procedure.

In Example 30, the subject matter of one or any combination of Examples 1-29 can optionally be configured such that the at least one cardiac function management device parameter has a value established using cardiac functional imaging information obtained from the fMRI device including information a deoxyhemoglobin.

In Example 31, the subject matter of one or any combination of Examples 1-30 can optionally be configured such that the at least one cardiac function management device parameter has a value established using cardiac functional imaging information obtained from the fMRI device including information an amount of blood flow.

In Example 32, the subject matter of one or any combination of Examples 1-31 can optionally be configured such that the at least one cardiac function management device parameter has a value established using cardiac functional imaging information obtained from the fMRI device including information about at least one of a deoxyhemoglobin level or an amount of blood flow.

In Example 33, the subject matter of one or any combination of Examples 1-32 can optionally be configured such that the establishing the value of the at least one cardiac function management device parameter comprises establishing the at least one cardiac function management device parameter associated with at least one of a decreased deoxyhemoglobin level indicated by the cardiac functional imaging information obtained from the fMRI device or an increased amount of blood flow indicated by the cardiac functional imaging information obtained from the fMRI device.

In Example 34, the subject matter of one or any combination of Examples 1-33 can optionally be configured such that the establishing the value of the at least one cardiac function management device parameter comprises: establishing an efficiency indicator value of the heart using at least one of the information about the deoxyhemoglobin level or amount of blood flow obtained from the fMRI device; and establishing the at least one cardiac function management device parameter associated with an increased efficiency indicator value.

In Example 35, the subject matter of one or any combination of Examples 1-34 can optionally be configured such that the at least one cardiac function management device parameter of the ambulatory medical device is configured to provide a correlation between a measurement of the ambulatory medical device to a functional physiological measurement obtained using an imaging device.

Example 36 can include, or can optionally be combined with the subject matter of one or any combination of Examples 1-35 to include subject matter (such as a method, a means for performing acts, or a device-readable medium including instructions that, when performed by the device, cause the device to perform acts), comprising: establishing a value of at least one cardiac function management device parameter, of an ambulatory medical device, at least in part using cardiac functional imaging information obtained from a fMRI device.

In Example 37, the subject matter of one or any combination of Examples 1-36 can optionally comprise instructions to provide a therapy to a subject using the at least one cardiac function management device parameter, and wherein the at least one cardiac function management device parameter comprises a cardiac function management therapy control parameter configured to control at least one of a therapy timing, a therapy energy level, or a therapy location.

In Example 38, the subject matter of one or any combination of Examples 1-37 can optionally comprise instructions such that the establishing the value of the at least one cardiac function management device parameter comprises using information obtained from an imaging apparatus including at least one of an echocardiogram, a computed tomography (CT) scan, a positron emission tomography (PET) scan, an MRI scan, or an X-ray image.

In Example 39, the subject matter of one or any combination of Examples 1-38 can optionally comprise instructions to establish operative functionality of the ambulatory medical device that is compatible for use during an MRI procedure and that allows cardiac signal sensing during the MRI procedure.

In Example 40, the subject matter of one or any combination of Examples 1-39 can optionally comprise instructions to vary the value of the at least one cardiac function management device parameter when a subject associated with the ambulatory medical device is undergoing an imaging procedure.

In Example 41, the subject matter of one or any combination of Examples 1-40 can optionally comprise instructions such that establishing the value of the at least one cardiac function management device parameter using the cardiac functional imaging information obtained from the fMRI device comprises using information about at least one of a deoxyhemoglobin level or an amount of blood flow.

In Example 42, the subject matter of one or any combination of Examples 1-41 can optionally comprise instructions such that establishing the value of the at least one cardiac function management device parameter comprises establishing the value of the at least one cardiac function management device parameter associated with at least one of a decreased deoxyhemoglobin level indicated by the cardiac functional imaging information obtained from the fMRI device or an increased amount of blood flood flow indicated by the cardiac functional imaging information obtained from the fMRI device.

In Example 43, the subject matter of one or any combination of Examples 1-42 can optionally comprise instructions such that the establishing the value of the at least one cardiac function management device parameter comprises: establishing an efficiency indicator value of the heart using at least one of the information about the deoxyhemoglobin level or amount of blood flow obtained from the fMRI device; and establishing the at least one cardiac function management device parameter associated with an increased efficiency indicator value.

In Example 44, the subject matter of one or any combination of Examples 1-43 can optionally comprise instructions such that the at least one cardiac function management device parameter of the ambulatory medical device is configured to provide a correlation between a measurement of the ambulatory medical device to a functional physiological measurement obtained using an imaging device.

Example 45 can include, or can optionally be combined with the subject matter of one or any combination of Examples 1-44 to include subject matter (such as a method, a means for performing acts, or a device-readable medium including instructions that, when performed by the device, cause the device to perform acts), comprising: establishing, using a processor circuit, a value of at least one of a cardiac function management device parameter, of an ambulatory medical device, at least in part using cardiac functional imaging information obtained from a fMRI device.

In Example 46, the subject matter of one or any combination of Examples 1-45 can optionally be performed such that the establishing the value of the at least one cardiac function management device parameter using cardiac functional imaging information obtained from the fMRI device comprises using information about at least one of a deoxyhemoglobin level or an amount of blood flow.

In Example 47, the subject matter of one or any combination of Examples 1-46 can optionally be performed such that the establishing the value of the at least one cardiac function management device parameter comprises establishing the value of the at least one cardiac function management device parameter associated with at least one of a decreased deoxyhemoglobin level indicated by the cardiac functional imaging information obtained from the fMRI device or an increased amount of blood flood flow indicated by the cardiac functional imaging information obtained from the fMRI device.

In Example 48, the subject matter of one or any combination of Examples 1-47 can optionally comprise establishing the at least one cardiac function management device parameter of the ambulatory medical device to provide a correlation between a measurement of the ambulatory medical device to a functional physiological measurement obtained using an imaging device.

These examples can be combined in any permutation or combination. This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of portions of a cardiac function management system and an environment in which it is used.

FIG. 2 shows an example in which a patient with an implantable cardiac rhythm management device connected to the heart, such as via one or more leads, is positioned for imaging by an imaging device.

FIG. 3 shows an example of using imaging information to adjust one or more device parameters of an implantable or other ambulatory device, such as the implantable cardiac function management device.

### DETAILED DESCRIPTION

### MRI Overview

Nuclear magnetic resonance (NMR) devices (e.g., an MRI scanner, an NMR spectrometer, or other NMR device) can produce both static and time-varying magnetic fields. For example, an MRI scanner can provide a strong static magnetic field, B₀, such as to align nuclei within a subject to the axis of the B₀ field. The B₀ can provide a slight net magnetization (e.g., a “spin polarization”) among the nuclei in bulk because the spin states of the nuclei are not randomly distributed among the possible spin states. Because the resolution attainable by NMR devices can be related to the magnitude of the B₀ field, a stronger B₀ field can be used to spin polarize the subject's nuclei to obtain finer resolution images. NMR devices can be classified according the magnitude of the B₀ field used during imaging, such as a 1.5 Tesla B₀ field, a 3.0 Tesla B₀ field, etc.

After nuclei are aligned using the B₀ field, one or more radio frequency (RF) magnetic excitation pulses can be delivered such as to alter the alignment of specified nuclei (e.g., within a particular volume or plane to be imaged within the subject). The power, phase, and range of frequencies of the one or more RF excitation pulses can be selected, such as depending on the magnitude of the B₀ field, the type or resonant frequency of the nuclei to be imaged, or one or more other factors. After the RF excitation pulses are turned off, one or more RF receivers can be used to detect a time-varying magnetic field (e.g., a flux) developed by the nuclei as they relax back to a lower energy state, such as the spin polarized state induced by the static magnetic field, B₀.

One or more gradient magnetic fields can also be provided during MR, such as to create a slight position-dependent variation in the static polarization field. The variation in the static polarization field slightly alters the resonant frequency of the relaxing nuclei, such as during relaxation after excitation by the one or more RF pulses. Using the gradient field along with the static field can provide “spatial localization” of signals detected by the RF receiver, such as by using frequency discrimination. Using a gradient field allows a volume or plane to be imaged more efficiently. In a gradient field example, signals received from relaxing nuclei can include energy in respective unique frequency ranges corresponding to the respective locations of the nuclei.

Active MRI equipment can induce unwanted torques, forces, or heating in an IMD or other conductive implant, or can interfere with operation of the IMD. In certain examples, the interference can include disruption in sensing by the IMD, interference in communication between the IMD and other implants or external modules during MRI operation, or disruption in monitoring or therapeutic function of the IMD.

During an MRI scan, the one or more RF excitation pulses can include energy delivered at frequencies from less than 10 MHz to more than 100 MHz, such as corresponding to the nuclear magnetic resonances of the subject nuclei to be imaged. The gradient magnetic field can include energy delivered at frequencies lower than the RF excitation pulses, because most of the AC energy included in the gradient field is provided when the gradient field is ramping or “slewing.” The one or more gradient magnetic fields can be provided in multiple axes, such as including individual time-varying gradient fields provided in each of the axes to provide imaging in multiple dimensions.

In an example, the static field, B₀, can induce unwanted forces or torques on ferromagnetic materials, such as steel or nickel. The forces or torques can occur even when the materials are not directly within the “bore” of the MRI equipment—because significant fields can exist near the MRI equipment. Moreover, if an electric current is switched on or off in the presence of the B₀ field, a significant torque or force can be suddenly imposed in the plane of the circulation of the current, even though the B₀ field itself is static. The induced force or torque can be minimal for small currents, but the torque can be significant for larger currents, such as those delivered during defibrillation shock therapy. For example, assuming the circulating current is circulating in a plane normal (e.g., perpendicular) to the static field, the torque can be proportional to the magnitude of the B₀ field, multiplied by the surface area of the current loop, multiplied by the current.

Time-varying fields, such as the gradient field or the field associated with the RF excitation pulse, can present different risks than the static field, B₀. For example, the behavior of a wire loop in the presence of a time-varying magnetic field can be described using Faraday's law, which can be represented by

$$ɛ = - \frac{d {Φ}_{{B}_{1}}}{d t} ,$$

in which ε can represent the electromotive force (e.g., in volts), such as developed by a time-varying magnetic flux. The magnetic flux can be represented as

$${Φ}_{B 1} = {\int }_{S} \int {B}_{1} \cdot d S ,$$

in which B₁ can represent an instantaneous magnetic flux density vector (e.g., in Webers per square meter, or Tesla). If B₁ is relatively uniform over the surface S, then the magnetic flux can be approximately Φ_{B1}=|B₁∥A|, where A can represent the area of the surface S. Operating MRI equipment can produce a time-varying gradient field having a slew rates in excess of 100 Tesla per second (T/s). The slew rate can be similar to a “slope” of the gradient field, and is thus similar to

$$\frac{d {Φ}_{{B}_{1}}}{d t} .$$

The electromotive force (EMF) of Faraday's law can cause an unwanted heating effect in a conductor—regardless of whether the conductor is ferromagnetic. EMF can induce current flow in a conductor (e.g., a housing of an IMD, one or more other conductive regions within an IMD, or one or more other conductive implants). The induced current can dissipate energy and can oppose the direction of the change of the externally applied field (e.g., given by Lenz's law). The induced current tends to curl away from its initial direction, forming an “eddy current” over the surface of the conductor, such as due to Lorentz forces acting upon electrons moving through the conductor. Because non-ideal conductors have a finite resistivity, the flow of induced current through the conductor can dissipate heat. The induced heat can cause a significant temperature rise in or near the conductor over the duration of the scan. The power dissipated by the eddy current can be proportional to the square of both the peak flux density and the frequency of the excitation.

Generally, induced currents, such as induced by the RF magnetic excitation pulse, can concentrate near the surface of a conductor, a phenomenon that can be referred to as the skin effect. The skin effect can limit both the magnitude and depth of the induced current, thus reducing power dissipation. However, the gradient field can include energy at a much lower frequency than the RF magnetic excitation field, which can more easily penetrate through the housing of the IMD. Unlike the field from the RF excitation pulse, the gradient field can more easily induce bulk eddy currents in one or more conductors within the IMD housing, such as within one or more circuits, capacitors, batteries, or other conductors.

Aside from heating, the EMF can create, among other things, non-physiologic voltages that can cause erroneous sensing of cardiac electrical activity, or the EMF can create a voltage sufficient to depolarize cardiac tissue or render the cardiac tissue refractory, possibly affecting pacing therapy. In an illustrative example, an IMD can be connected to one or more leads, such as one or more subcutaneous or intravascular leads positioned to monitor the patient, or to provide one or more therapies to the patient. In this illustrative example, a surface area of a “circuit” including the lead, the housing of the IMD, and a path through at least partially conductive body tissue between an electrode on the lead and the IMD housing can be more than 300 square centimeters, or more than 0.03 square meters. Thus, using Faraday's law, the electromotive force (EMF) developed through the body tissue between the electrode (e.g., a distal tip or ring electrode) of the lead and the housing of the IMD can be more than 0.03 square meters times 100 t/s, or more than 3 volts.

### System Overview

The present inventors have recognized, among other things, that an implantable or other ambulatory medical device, such as a pacer, defibrillator, or other cardiac rhythm management device, can use functional magnetic resonance imaging (fMRI) information, such as for helping optimize one or more parameters of the implantable or other ambulatory device.

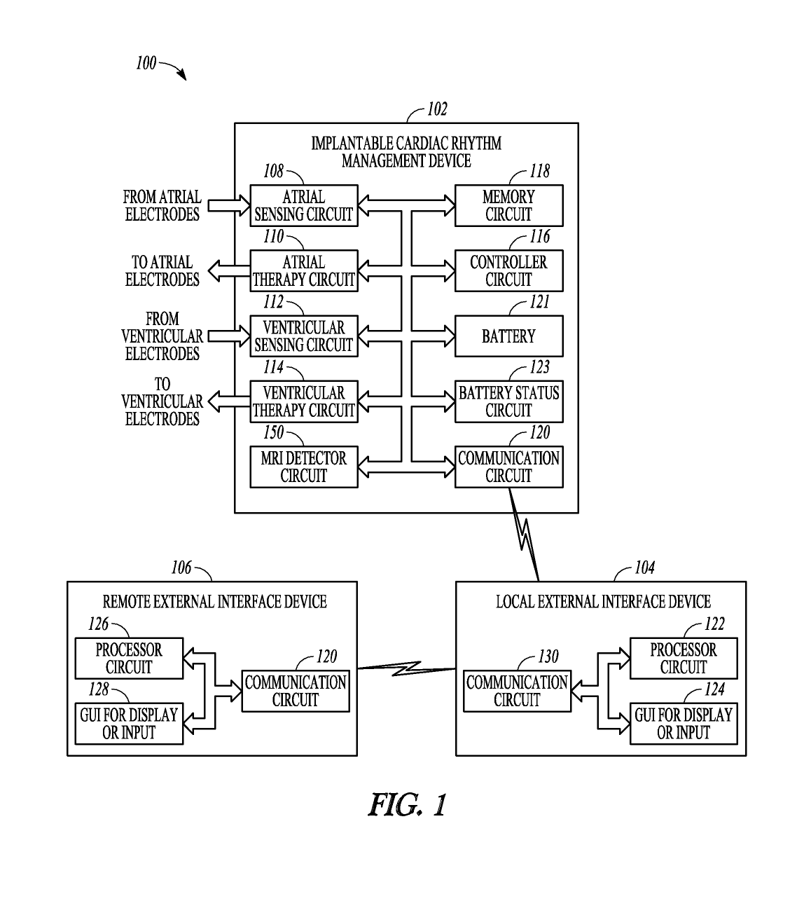

FIG. 1 illustrates an example of portions of a cardiac function management system 100 and an environment in which it is used. In an example, the system 100 can include an implantable cardiac function management device 102, a local external interface device 104, and an optional remote external interface device 106. In an example, the implantable device 102 can include an atrial sensing circuit 108, an atrial therapy circuit 110, a ventricular sensing circuit 112, a ventricular therapy circuit 114, a controller circuit 116, a memory circuit 118, a communication circuit 120, a power source such as a battery 121, and a battery status circuit 123.

The atrial sensing circuit 108 can be coupled to electrodes, such as an intra-atrial electrode or any other electrode that can permit sensing of an intrinsic atrial cardiac signal including atrial depolarization information. The atrial therapy circuit 110 can be similarly coupled to these or other electrodes, such as for delivering pacing, cardiac resynchronization therapy (CRT), cardiac contractility modulation (CCM) therapy, defibrillation cardioversion shocks, or other energy pulses to one or both atria.

The ventricular sensing circuit 112 can be coupled to electrodes, such as an intra-ventricular electrode or any other electrode that can permit sensing of an intrinsic ventricular cardiac signal including ventricular depolarization information. The ventricular therapy circuit 114 can be similarly coupled to these or other electrodes, such as for delivering pacing, cardiac resynchronization therapy (CRT), cardiac contractility modulation (CCM) therapy, defibrillation cardioversion shocks, or other energy pulses one or both ventricles.

A controller circuit 116 can be coupled to the atrial sensing circuit 108 and the ventricular sensing circuit 112 such as to receive information from the sensed cardiac signals, and can be coupled to the atrial therapy circuit 110 and the ventricular therapy circuit 114 such as to provide control or triggering signals such as to trigger timed delivery of the therapy pulses. In an example, the controller circuit 116 can be configured to provide control to help permit the therapy to be effectively delivered, such as in combination with one or more other therapies (e.g., bradycardia pacing, antitachyarrhythmia pacing (ATP), cardiac resynchronization therapy (CRT), atrial or ventricular defibrillation shock therapy) or functionalities (e.g., autothreshold functionality for automatically determining pacing threshold energy, autocapture functionality for automatically adjusting pacing energy to capture the heart, etc.). In an example, this can include providing dedicated modules within the controller circuit 116, or providing executable, interpretable, or otherwise performable code configure the controller circuit 116.

A memory circuit 118 can be coupled to the controller circuit 116, such as to store control parameter values, physiological data, or other information. A communication circuit 120 can be coupled to the controller circuit 116 such as to permit radiofrequency (RF) or other wireless communication with an external device, such as the local external interface device 104 or the remote external interface device 106.

In an example, the battery 121 can include one or more batteries to provide power for the implantable device 102. In an example, the battery 121 can be rechargeable, such as by wireless transcutaneous power transmission from an external device to the implantable device 102. The battery status circuit 123 can be communicatively coupled to each of the battery 121 and the controller circuit 116, such as to determine battery status information, for example, indicative of how much energy remains stored in the battery 121. The controller circuit 116 can be configured to alter operation of the implantable device 102, such as based at least in part on the battery status information.

The local external interface device 104 can include a processor circuit 122 and a graphic user interface (GUI) 124 or like device such as for displaying information or receiving user input as well as a communication circuit 130, such as to permit wired or wireless communication with the remote external interface device 106 over a communications or computer network. Similarly, the remote external interface device 106 can include a processor circuit 126 and a graphic user interface (GUI) 128 or like device such as for displaying information or receiving user input as well as a communication circuit 132, such as to permit wired or wireless communication with the local external interface device 104 over the communications or computer network. Because the system 100 can include processing capability in the implantable device 102 (e.g., provided by the controller circuit 116), the local external interface device 104 (e.g., provided by the processor circuit 122), and the remote external interface device 106 (e.g., provided by the processor circuit 126), various methods discussed in this document can be implemented at any of such locations, or tasks can be distributed between two or more of such locations.

### Device Adjustment or Optimization Examples

Imaging devices can be used to obtain imaging information such as cardiac imaging information. The information obtained from an imaging device can be used to provide one or more indications relating to cardiac function. Examples of such indications can include, but are not limited to, localized levels of deoxyhemoglobin (e.g., as an indication of oxygenation levels in certain tissues or blood), amounts of blood flow, motion of one or more portions of a heart (e.g., a valve opening or closure, a cardiac wall motion such as a septal wall motion a freewall motion or other cardiac wall motion, etc.), diastolic or systolic activity of one or more portions of the heart (e.g., filling activity, or ejection activity of the heart, or one or more other parameters), or cardiac metabolic activity (e.g., cardiac metabolic uptake of a biologically active tracer molecule, such as used in positron-emission tomography (PET)).

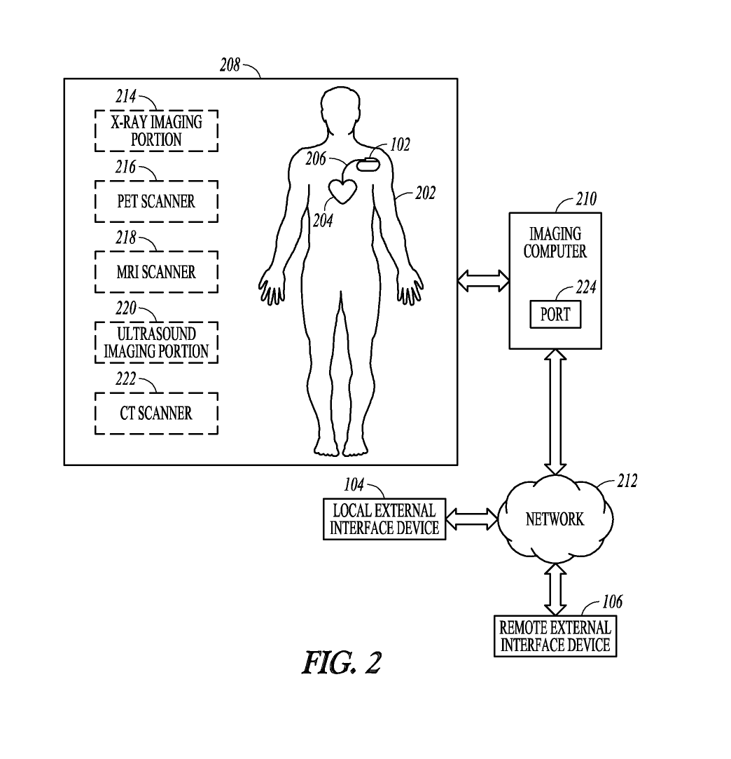

FIG. 2 illustrates generally an example that can include an implantable cardiac function management device 102 that can be connected or coupled to the heart 204 of a patient 202, such as via one or more leads 206. The patient 202 can be positioned for imaging by an imaging apparatus 208. The imaging apparatus 208 can be configured to perform one or more imaging operations, such as by using one or more imaging modalities or techniques. Such imaging techniques can include using one or more imaging modalities, such as one or more of an X-ray imaging portion 214, a positron emission tomography (PET) scanner 216, a MRI scanner 218, an ultrasound imaging portion 220, a computed tomography (CT) scanner 222, or other imaging modality. Examples of combinations of such imaging techniques or modalities, such as performed by the imaging apparatus 208 can include a combination PET-CT scanner, a combination PET-MRI scanner, or one or more other combinations.

The imaging apparatus 208 can be communicatively coupled to an imaging computer 210, such as by using a port 224. In an example, the imaging computer 210 can be configured to do one or more of: provide one or more control signals to operate the imaging apparatus 208, receive imaging data, signal-process the received imaging data, or store or display the signal-processed received imaging data or information derived therefrom. In an example, the raw or signal-processed imaging data can be communicated, such as via a computer or communications network 212, such as to a processor capable of determining one or more indications related to cardiac function. In an example, such processing can be performed at the remote external interface device 106, at the local external interface device 104, or at the imaging computer 210.

The cardiac imaging information can be used, such as to establish, adjust, optimize, or otherwise determine a device parameter of an implantable or other ambulatory medical device, such as the implantable cardiac function management device 102. Such device parameters that can be affected by the cardiac imaging information can include a device therapy parameter, a device diagnostic parameter, or other device operational parameter.

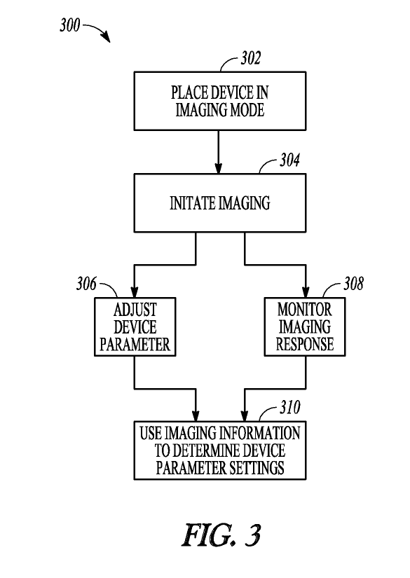

FIG. 3 shows an example 300 of using imaging information to adjust one or more device parameters of an implantable or other ambulatory device, such as the implantable cardiac function management device 102. In an example, the one or more device parameters of the implantable or other ambulatory device that can be adjusted can include one or more therapy control parameters, such as can including one or more of a therapy timing parameter, a therapy energy level parameter, or a therapy location parameter.

At 302, the implantable cardiac function management device 102 can be placed in an imaging mode. In an example, the imaging mode can include an MRI mode, such as to establish operative functionality that is compatible for use during an MRI procedure, or during exposure to one or more other imaging techniques to be used during imaging of the subject with the implantable cardiac function management device 102. For example, the operative functionality of the MRI mode can be that which is configured to be less susceptible to disruption during an imaging procedure than a normal mode of operation would be when undergoing such an imaging procedure, wherein the normal mode of operation is configured for use when not undergoing MRI scanning or other imaging procedures. In an example, the implantable cardiac function management device 102 can be transitioned from the normal non-MRI mode to the MRI mode, such as by remote programming using the remote external interface device 106 or by local programming such as by using the local external interface device 104, such as before the patient is placed in the imaging apparatus 208. In an example, during the MRI mode, an MRI-safe set of therapy control and other device parameters can be provided.

In an example, the implantable cardiac function management device 102 can include an MRI detector circuit 150, such as for automatically detecting when the implantable cardiac function management device 102 is located in or near the bore of the MRI scanner 218 undergoing imaging and, in response, can automatically transition from the normal non-MRI mode to the MRI mode, such as without requiring user intervention to initiate such transition. In an example, the MRI detector circuit 150 can include a reed switch, such as to detect the presence of a magnetic field indicative of an MRI scanner performing an MRI scanning operation nearby, such as to automatically transition from the normal non-MRI mode to the MRI mode or to prompt a user to do so.

In an example, the MRI detector circuit 150 can include a Hall-effect sensor, such as to detect the presence of an MRI field indicative of an MRI scanner performing an MRI scanning operation nearby, such as to automatically transition from the normal non-MRI mode to the MRI mode or to prompt a user to do so. An example of using a Hall effect sensor in an implantable medical device to sense a magnetic field is described in Linder et al. U.S. Patent Pub. No. 2009/0157146, entitled IMPLANTABLE MEDICAL DEVICE WITH HALL SENSOR, assigned to Cardiac Pacemakers, Inc., which is incorporated herein by reference in its entirety, including its description of using a Hall-effect sensor to detect a magnetic field, such as that of an MRI scanner. An example of using a Hall-effect sensor in conjunction with an MRI operating mode of an implantable medical device is described in Cooke et al. U.S. Patent Pub. No. 2009/0138058, entitled MRI OPERATION MODES FOR IMPLANTABLE MEDICAL DEVICES, which is assigned to Cardiac Pacemakers, Inc., which is incorporated herein by reference in its entirety, including its description of using a Hall-effect sensor in conjunction with an MRI operating mode of an implantable medical device.

In an example, the MRI detector circuit 150 can additionally or alternatively include an inductor saturation detector, such as to detect the presence of an MRI field indicative of an MRI scanner performing an MRI scanning operation nearby, such as to automatically transition from the normal non-MRI mode to the MRI mode or to prompt a user to do so. An example of using inductor saturation to perform MRI detection is described in Stessman, U.S. Pat. No. 7,509,167, entitled MRI DETECTOR FOR AN IMPLANTABLE MEDICAL DEVICE, assigned to Cardiac Pacemakers, Inc., which is incorporated herein by reference in its entirety, including its description of using inductor saturation to perform MRI detection. Such MRI detection techniques, methods, or apparatuses can be used to place the device 102 in an MRI-safe mode when the device 102 is otherwise exposed to other environmental magnetic fields that might disrupt the operation of the device 102 (e.g., during one or more other imaging procedures, or when the device 102 is in proximity to heavy equipment or laboratory equipment that can generate a disruptive magnetic field).

At 304, imaging can be initiated. At 306, at least one device parameter, such as a diagnostic parameter, a therapy parameter, or other operational parameter can be established ore adjusted. For example, a therapy control parameter (e.g., a pacing rate, an electrostimulation timing parameter, an atrioventricular (AV) delay, a bi-ventricular electrode selection, an interventricular (VV) delay, intraventricular delay, a pacing mode, a neurostimulation parameter, a drug titration parameter, etc.) can be varied, such as while the imaging response is concurrently monitored at 308, after the imaging response is monitored, or in between successive imaging operations. At 310, the imaging information (e.g., such as visualization or functional physiological information obtainable from the imaging modality) can be used to determine one or more device parameter settings, such as including a therapy control parameter setting, a diagnostic parameter setting, or another device operational parameter setting.

fMRI Device Adjustment or Optimization Examples

Functional magnetic resonance imaging (fMRI) can be used to obtain functional physiological information, such as information indicating the localized levels of deoxyhemoglobin of certain tissue or blood. Deoxyhemoglobin levels can indicate oxygenation levels of the imaged tissue or blood. For example, because deoxyhemoglobin is a hemoglobin molecule that has “released” its oxygen, an increase in deoxyhemoglobin levels can indicate a decrease in oxygenation levels of the tissue or blood. Conversely, a decrease in deoxyhemoglobin levels can indicate an increase in oxygenation levels of the tissue or blood. Deoxyhemoglobin levels can also be used to provide information about blood flow. For example, as the amount of blood flow increases, deoxyhemoglobin molecules can become diluted in the larger volume of blood. Such dilution can cause a decrease in deoxyhemoglobin levels, such as indicating an increased amount of blood flow. Similarly, an increase in deoxyhemoglobin levels can indicate a decreased amount of blood flow.

In one fMRI approach, techniques using fMRI can be applied to brain imaging to provide information about oxygenation or blood flow in localized regions of the brain. The present inventors have recognized, among other things, that fMRI functional physiological information can be used to adjust a parameter of a medical device, such as the implantable cardiac function management device 102. For example, such fMRI functional physiological information can be used to provide information about oxygenation levels or blood flow in the heart, from which it can be inferred how much oxygen is being used to “fuel” the heart. This information, in turn, can be combined with information or assumptions about how much work the heart is doing (e.g., as inferred by how much cardiac output it is producing), such as to obtain a measure of how efficiently the heart is operating.

Such oxygenation information or efficiency information can be used to adjust a device parameter. For example, such information can be used as a feedback parameter that can be monitored, such as to control or help control operation of an implantable or other ambulatory device, such as a cardiac function management device 102. In an example, if one or more device parameters of the cardiac function management device 102 can be varied during fMRI monitoring, then the resulting oxygenation or efficiency information can be used (alone, or with one or more other indicators of cardiac function) to determine what settings should be applied to the diagnostic, therapy, or other device parameters of the cardiac function management device 102 (or, e.g., in what direction such parameters should be adjusted, such as to improve cardiac function).

In an example, fMRI cardiac functional imaging information can be used to optimize or otherwise determine one or more device parameters, such as a therapy control parameter, of an implantable or other ambulatory device, such as the implantable cardiac function management device 102.

In an example, one or more device parameters can optionally be varied, and one or more response variables (including at least one response variable using fMRI information, such as functional physiological information that can be obtained from such imaging) can be concurrently monitored. In an example, one or more device parameters can optionally be varied, and one or more response variables can be monitored in between successive imaging operations during the same or different imaging sessions. In an example, the one or more device parameters that can be varied can include one or more therapy control parameters, such as is described above. An example of varying therapy control parameters and monitoring at least one response variable is described in Dong et al. U.S. patent application Ser. No. 12/249,856 entitled METHOD AND APPARATUS TO TREND AND OPTIMIZE AN IMPLANTABLE MEDICAL DEVICE USING A PATIENT MANAGEMENT SYSTEM, which was filed on Oct. 10, 2008, and which is assigned to Cardiac Pacemakers, Inc., and which published on Apr. 23, 2009 as U.S. Patent Pub. No. 2009-0105777-A1, which is incorporated by reference herein in its entirety, including its description of varying a therapy control parameter and monitoring at least one response variable. Another example of varying therapy control parameters and monitoring at least one response variable is described in Sathaye et al. U.S. patent application Ser. No. 11/614,578 entitled METHOD AND APPARATUS TO IMPLEMENT MULTIPLE PARAMETER SETS IN AN IMPLANTABLE DEVICE, which was filed on Dec. 21, 2006, and which is assigned to Cardiac Pacemakers, Inc., and which published on Jun. 26, 2008 as U.S. Patent Pub. No. 2008-0154323-A1, which is incorporated by reference herein in its entirety, including its description of varying a therapy control parameter and monitoring at least one response variable.

In addition to the techniques described in Dong et al. U.S. patent application Ser. No. 12/249,856 and Sathaye et al. U.S. patent application Ser. No. 11/614,578, the present inventors have recognized, among other things, that the implantable or other ambulatory device can use a subset of functionality deemed MRI-compatible or otherwise safe for use during one or more imaging procedures, such as an MRI imaging procedure. The present MRI-mode can, for example, allow at least some sensing capability for detecting cardiac electrical activity during imaging, to provide feedback during the adjustment of the one or more device parameters. Stubbs et al. U.S. Provisional Patent Application No. 61/291,309, entitled SENSING DURING MAGNETIC RESONANCE IMAGING, which was filed on Dec. 30, 2009, and which is incorporated herein by reference in its entirety, provides an example of cardiac signal sensing that can be carried out during an MRI scanning procedure.

In an example, a brief blanking period can be triggered in response to gradient magnetic field detection so that unwanted signals or artifacts induced by the gradient magnetic fields or RF bursts can be ignored or suppressed during the period of imaging activity or scanning, but cardiac activity signals can still be sensed during gaps between such blanking periods.

In an example, fMRI data can provide functional physiological information such as information about local oxygenation of the heart 204, such as a composite representation of overall oxygenation of the heart, or more specific information about oxygenation of a desired or specified region of the heart 204. Without being bound by theory, it is believed that an fMRI indication of greater oxygenation of the heart can be indicative of less oxygen usage by the cardiac muscle in providing heart contractions. Without being bound by theory, it is believed that it can be beneficial to provide cardiac rhythm management, neurostimulation, drug, or other therapy in such as way as to allow the heart to operate in a manner that uses less oxygen, such as while otherwise maintaining a desired level of cardiac output or cardiac performance (e.g., such as can be indicated by an ejection fraction, dP/dt, blood flow, or one or more other parameters indicative of cardiac output or cardiac performance).

In an example, a processor or controller circuit can be used to select or adjust one or more cardiac function management or neurostimulation parameters, such as to generally decrease or minimize the deoxyhemoglobin level indication (e.g., increase or maximize the oxygenation indication) provided by the fMRI. In such a way, fMRI-indicated cardiac oxygenation information can be used as the sole response variable for selecting or adjusting one or more therapy, diagnostic, or other operational device parameters, or the fMRI-indicated cardiac oxygenation information can be used as one of several response variables (e.g., fMRI or non-fMRI) for selecting or adjusting one or more therapy, diagnostic, or other device parameters.

In an example, fMRI functional physiological data can provide information about how much work is being performed by the heart 204. In an example, the cardiac output represents the amount of blood being expelled by the heart. The cardiac output can be determined using fMRI blood flow information or other blood flow information, such as from a blood flow meter or the like. The cardiac output can be taken as an indication of how much work is being done by the heart. In such a way, fMRI work information can be used as a response variable, such as for selecting or adjusting one or more therapy control or other device parameters. In an example, the one or more device parameters, such as the therapy control parameters, can be set in such a way so as to generally increase or maximize the amount of work being performed by the heart, such as during an otherwise stable period of metabolic demand (e.g., with a reclining patient, at rest, or during one or more steady-state or other periods of activity or posture that allow comparison to other like steady-state periods of activity or posture).

In an example, fMRI information about (1) how much fuel (e.g., oxygen) is being consumed by the heart 204 (e.g., using the fMRI or other oxygenation information) and (2) how much work is being performed by the heart 204 (e.g., using an indicator of work such as cardiac output, such as can be determined using fMRI blood flow or other information), can be used together to compute a measure of efficiency of heart pumping. For example, if the value of the oxygenation indicator (e.g., deoxyhemoglobin level indicated by the fMRI information) of the heart 204 remains constant, an increase in the value of the work indicator (e.g., the blood flow indicated by the fMRI information) of the heart 204 can correlate to a higher efficiency indicator value. In such an example, a decrease in the value of the work indicator of the heart 204 can correlate to a lower efficiency indicator value. Similarly, if the value of the work indicator of the heart 204 remains constant, an increase in the value of the oxygenation indicator of the heart 204 (e.g., a decrease in the deoxyhemoglobin level indicated by the fMRI information) can correlate to a higher efficiency indicator value, while a decrease in the value of the oxygenation indicator can correlate to a lower efficiency indicator value. In such a way, fMRI efficiency information can be used as a response variable, such as for selecting or adjusting one or more therapy control or other device parameters. In an example, the one or more device parameters, such as the therapy control parameters, can be set in such a way so as to generally increase or maximize the efficiency of work being performed by the heart, such as during an otherwise stable period of metabolic demand (e.g., with a reclining patient, at rest, or during one or more steady-state or other periods of activity or posture that allow comparison to other like steady-state periods of activity or posture).

The above examples have discussed efficiency with particular emphasis on work performed (e.g., using fMRI or other cardiac output information) relative to fuel consumed (e.g., using fMRI or other oxygenation information). However, the work performed can be indicated by a surrogate indicator for cardiac performance, which can be obtained from fMRI or other information, and which can optionally be taken relative to an fMRI or other indication of a cardiac input. For example, an “efficiency” indication of cardiac output or cardiac performance relative to a cardiac input can be determined using (1) cardiac contractility information (e.g., dP/dt, such as using a pressure sensor, cardiac wall motion, such as using an intracardiac impedance or MRI imaging information), which can be taken relative to cardiac input, such as fuel consumed (e.g., using fMRI or other oxygenation information).

The above examples have discussed, among other things, using fMRI information to select or adjust a diagnostic, therapy, or other operational device parameter, such as either during, after, or between imaging sessions, such as a fMRI imaging session, with emphasis on selecting or adjusting a therapy control parameter such as by using fMRI information as feedback. However, fMRI functional physiological information can be used for various purposes, including selecting or adjusting a diagnostic device parameter. In an example, a medical device, such as the implantable cardiac function management device 102, can be used to detect or diagnose a physiological condition, for example, coronary ischemia. For example, the implantable cardiac function management device 102 can sense a cardiac electrogram, such as by using the ventricular sensing circuit 112, from which a morphological characteristic such as ST segment elevation above a baseline value can indicate the occurrence of coronary ischemia or even a myocardial infarction (“heart attack”). Such information can be stored or used to generate an alert or other indication, such as can be communicated to a user or automated process. In an example, fMRI functional physiological information such as blood oxygenation (or deoxygenation) of the subject's coronary arteries or myocardium can be detected, such as using an fMRI scanner. Such information can be used with the ST segment information, such as to augment, validate, calibrate, or otherwise modify the use of the ST segment information or a physiological status (e.g., ischemia, MI, etc.) that can be derived therefrom.

In a calibration example, baseline fMRI functional physiological information such as blood oxygenation (or deoxygenation) of the subject's coronary arteries or myocardium can be detected, such as using an fMRI scanner. Then, a cardiac stress condition such as ischemia can be induced in the subject, such as by administering a drug (e.g., dobutamine or adenosine) or performing a physical activity stress test. During both the baseline and stress conditions, the implantable cardiac function management device 102 can be used, such as to measure one or more physiological parameters (e.g., an ST segment elevation of a sensed electrogram), or optionally to adjust one or more cardiac function management therapy parameters. A correlation can be computed between a baseline-to-stress change in the fMRI functional physiological information (e.g., blood oxygenation or deoxygenation) and a corresponding baseline-to-stress change in the physiological information measured by the implantable cardiac function management device 102 (e.g., ST segment elevation). Such correlation can be used to calibrate a later change in the physiological information (e.g., ST segment elevation) measured by the implantable cardiac function management device 102. For example, this correlation can permit the later change in the physiological information (e.g., ST segment elevation) measured by the implantable cardiac function management device 102 to be expressed in terms of the units of the fMRI functional physiological information (e.g., oxygenation or deoxygenation). To create the correlation, a single stress state relative to a baseline can be used, or multiple (e.g., different) stress states can be used, such as to create a correlation look-up table or function. If a good enough correlation exists between the physiological information measured by the implantable cardiac function management device 102 and the fMRI functional physiological information, such correlation can even be used to create an implantably (e.g., chronic ambulatory) obtainable surrogate for the non-implantable, non-ambulatory fMRI functional physiological measurement. Although the above example has described the correlation with an emphasis on calibrating an implantable measurement using fMRI functional physiologic information, calibration of the fMRI functional physiologic information using the implantably-obtained information can also be performed.

### MRI Device Adjustment or Optimization Examples

Magnetic resonance imaging (MRI) scanners, such as the MRI scanner 218 can be used to obtain cardiac imaging visualization information (such cardiac imaging visualization information is also obtainable using an fMRI scanner 218, so the techniques described herein using cardiac imaging visualization information should be understood to apply in that context as well). Cardiac imaging visualization information obtained from an MRI scanner 218 can be used to provide indications about cardiac function, such as information about one or more of the motion or acceleration of one or more portions of the heart 204, or diastolic or systolic activity of one or more portions of the heart 204.

In an example, MRI cardiac imaging visualization information can be used to select, establish, adjust, optimize, or otherwise determine one or more device parameters, such as a diagnostic parameter or a therapy control parameter or another operational device parameter of an implantable or other ambulatory device, such as the implantable cardiac function management device 102. In an example, one or more device parameters can optionally be varied, and one or more response variables (including at least one response variable using MRI visualization information) can be concurrently monitored. In an example, one or more device parameters can optionally be varied, and one or more response variables can be monitored between successive imaging operations during the same or different imaging sessions. In an example, the one or more device parameters that can be varied can include one or more therapy control parameters, such as described or incorporated above. This can include using or transitioning the implantable cardiac function management device into an MRI-mode or other imaging mode, such as described or incorporated above.

In an example, MRI cardiac visualization information can be used to measure or analyze dimension (e.g., size), position, or motion (e.g., amount, rate, rate of change (e.g., acceleration), etc.) of one or more portions of the heart 204. For example, MRI information can be used to provide one or more indications of cardiac wall motion, such as a septal wall motion. Patients suffering from congestive heart failure (CHF) generally have uncoordinated mechanical activity of the heart. For example, in a heart failure patient, myocardial depolarization and contraction of the right and left ventricle may not occur in synchrony with each other. Such unsynchronized or uncoordinated contractions can cause, among other things, decreased cardiac output, pulmonary or peripheral fluid accumulation, poor exercise tolerance, or other symptoms. In an example, septal wall motion of the heart 204 can be monitored, such as by using MRI cardiac imaging visualization information, and such information can be used with appropriate image processing to provide an indication of the coordination between contractions of the left and right ventricles.

Without being bound by theory, septal wall motion can be induced or affected, in an example, by a difference in pressure between the left and right ventricles. The difference in pressure between the left and right ventricles can be smaller in magnitude during a coordinated contraction (e.g., such as where the left and right ventricles contract simultaneously) than during an uncoordinated contraction (e.g., such as where the left and right ventricles do not contract simultaneously).

For example, if the right ventricle begins to contract before the left ventricle, the pressure in the right ventricle can be greater than the pressure in the left ventricle during the time that the right ventricle is contracting and the left ventricle has not yet begun to contract. In such an example, the difference in pressure between the ventricles can cause the septal wall to move, such as toward the left ventricle. Similarly, if the right ventricle finishes its contraction before the left ventricle, the pressure in the left ventricle can be greater than the pressure in the right ventricle during the time that the left ventricle is contracting and the right ventricle is no longer contracting. This difference in pressure between the ventricles can cause the septal wall to move, such as toward the right ventricle.

If, however, the left and right ventricles contract with proper coordination, the pressure differences between the ventricles can be reduced (but not necessarily eliminated, since LV pressures generally exceed RV pressures such as due to the different systemic fluid resistances that must be overcome). Reducing the pressure differences between the ventricles can reduce the movement of the septal wall. In an example, cardiac imaging visualization information obtained from an MRI device, such as the MRI device 218, can be used to indicate motion of the septal wall of the heart 204.

In an example, the amount septal wall motion of the heart 204 during a contraction can be determined using the MRI cardiac imaging visualization information, such as by one or more of a doctor, a nurse, a technician, or the like. For example, the movement of the septal wall can be determined such as by estimating the movement while viewing the MRI cardiac imaging information on a display.

In an example, the amount of septal wall motion of the heart 204 during a contraction can be determined such as by using automated image processing software that can provide instructions that can be stored and performed by a processor circuit or a controller circuit or the like. For example, multiple MRI cardiac images of the heart 204 can be captured at known increments of time. The images can be processed by the image processing software, such as to align the images and to detect the position of the septal wall in each image. For example, the image can be digitized, and the boundaries of the endocardium and the epicardium of the heart 204 in each image can be determined, such as by using circular arc filters to find the center point of the left ventricle and the approximate position of the epicardial and endocardial boundaries. The position of the septal wall within each image can be determined, such as by obtaining a first approximation of the position of the septal wall in the image using a mean filter to obtain a two dimensional graph of mean brightness across the image. The approximate position of the septal wall can be used as a region of interest in the image to conduct a more refined search, such as by moving Laplacian filters across the approximate position of the septal wall. The location of the maximum of each Laplacian filter can be determined, such as to estimate the position of the septal wall in each image. An illustrative non-limiting example of using imaging processing software to detect the position of a septal wall from an image is described in Geiser et al. U.S. Pat. No. 6,708,055, entitled METHOD FOR AUTOMATED ANALYSIS OF APICAL FOUR-CHAMBER IMAGES OF THE HEART, which is incorporated herein by reference in its entirety, including its description of using image processing software to detect the position of a septal wall from an image.

In an example, the indication of septal wall motion obtained from the MRI cardiac imaging visualization information can represent one or more of a displacement of the septal wall, the velocity of the septal wall with respect to time, or the acceleration of the septal wall with respect to time. For example, multiple cardiac images of the heart 204 can be taken at known increments of time, and the change in the position of the septal wall during a contraction can be determined such as by comparing the position of the septal wall in each of the MRI cardiac images. In an example, the maximum change in position of the septal wall during a contraction can be determined, such as by comparing the position of the septal wall at the end of ventricular diastole (e.g., when the ventricles have relaxed) to the position of the septal wall during ventricular systole (e.g., when the ventricles are contracting) and determining the maximum change in displacement of the septal wall during the contraction (or some other extremum, either for an individual contraction, or for an ensemble of multiple contractions, such as by determining a central tendency of such measured extrema). In such an example, the indication of velocity of the septal wall with respect to time (and the maximum value of the indication achieved during a contraction) can be determined such as by using the change in displacement of the septal wall and the known increment of time between cardiac images. Similarly, the acceleration of the septal wall (and the maximum value of the indication achieved during a contraction) can be determined such as by using the change in the velocity of the septal wall and the known increment of time between cardiac images.

In an example, MRI cardiac imaging visualization information can be used to provide an indication of cardiac valve motion, such as one or more of displacement, velocity, or acceleration of tissue corresponding to a valve opening or closure. In an example, the MRI cardiac imaging visualization information can be used to provide an indication of cardiac valve prolapse, such as mitral valve prolapse, or one or more other physiologic conditions. As discussed above with respect to septal wall motion, cardiac valve motion can be measured or analyzed such as using one or more of an automated image processing software, or a doctor, a nurse, a technician, or the like, such as to provide one or more indications of cardiac function.

In an example, MRI cardiac imaging visualization information can be used to provide an indication of one or more of diastolic or systolic activity of one or more portions of the heart 204. For example, using the MRI cardiac imaging visualization information, a change in the size of one or more of the atria or one or more of the ventricles during diastole or systole can be determined, such as by one or more of an automated image processing software, a doctor, a nurse, a technician, or the like. In an example, the change in size of one or more of the atria or one or more of the ventricles during diastole or systole can be determined using the difference in size of respective particular chamber between pre- and post-filling, or between successive filling cycles that can optionally use different sets of device parameters, or both. A greater change in the size of the chambers during diastole can indicate an increased filling activity of the chamber (e.g., increased diastolic activity). Similarly, a greater change in the size of the chambers during systole can indicate an increased ejection volume of the chamber during contraction (e.g., increased systolic activity).

In an example, one or more of the indications of septal wall motion (e.g., a displacement, a velocity, an acceleration, etc.), a cardiac valve motion (e.g., a valve prolapse), or diastolic or systolic activity (e.g., a preload or an ejection volume) obtained from the MRI cardiac imaging visualization information can be used, alone or in combination with each other or with other information, such as to establish, adjust, optimize, or otherwise determine one or more device parameters, such as one or more diagnostic parameters, one or more therapy control parameters, or one or more other operational parameters of an implantable or other ambulatory device, such as the implantable cardiac function management device 102. For example, one or more device therapy parameters (e.g., pacing rate, electrostimulation timing parameter, atrioventricular (AV) delay, bi-ventricular electrode selection, interventricular (VV) delay, intraventricular delay, pacing mode, neurostimulation parameter, drug titration parameter etc.) can optionally be varied, and one or more of the indications of one or more of septal wall motion, cardiac valve motion, diastolic activity, or systolic activity can be concurrently monitored, such as for use in a closed-loop of other feedback scheme.

In an example, the one or more device parameters can be adjusted such as to generally decrease or minimize the value of the indication of septal wall motion. For example, a VV delay can be varied in response to the indicated septal wall motion (alone, or in combination with one or more other measured physiological indicators), such as to help induce a more coordinated contraction or to help stabilize the pressures in the ventricles during contraction. In an example, the one or more device parameters can be adjusted such as to generally decrease or minimize an indication of cardiac valve prolapse. For example, one or more timing parameters such as AV delay, VV delay, or intraventricular delay can be adjusted such as to help reduce the pressure on a chamber that is causing a valve prolapse. In an example, the one or more device parameters can be adjusted such as to generally increase or maximize one or more of the indications of one or more of diastolic or systolic activity.

In an example, the one or more device parameters can be cycled through a specified list of parameters. In an example, a desired set of “optimal” device parameters can be determined using just one of the indications of septal wall motion, cardiac valve motion, diastolic activity, or systolic activity. In an example, the optimal device parameters can be chosen using one or more of the indications of septal wall motion, cardiac valve motion, diastolic activity, or systolic activity. For example, one or more of the indications of septal wall motion, cardiac valve motion, diastolic activity, or systolic activity can be combined, such as in a weighted composite index, which can be used to adjust one or more device parameters such as to generally increase or maximize the weighted composite index indicating how well the heart is functioning.

In an example, the weighted composite index can be determined such as by weighting the individual indications (e.g., septal wall motion, cardiac valve motion, diastolic activity, or systolic activity) according to an expected or measured relative contribution of the individual indication to an indication of overall cardiac function. For example, if an indication of coordinated contractions is expected or determined to be more indicative of how well the heart is functioning than an indication of ejection volume, the indication of septal wall motion can be given a greater weight than an indication of systolic activity in the calculation of the weighted composite index. In an example, the weighted composite index can be determined such as by using an average or other central tendency of the individual indications of cardiac function. In an example, the weighted composite index can be determined such as by using a ratio or other relative indication of the individual indications of cardiac function.

The above examples have discussed, among other things, using MRI visualization information to select or adjust a diagnostic, therapy, or other operational device parameter, such as either during, after, or between imaging sessions, such as an MRI imaging session, with emphasis on selecting or adjusting a therapy control parameter such as by using MRI visualization information as feedback. However, MRI visualization information can be used for various purposes, including selecting or adjusting a diagnostic device parameter. In an example, a medical device, such as the implantable cardiac function management device 102, can be used to detect or diagnose a physiological condition, for example, cardiac wall motion determined from a transcardiac or intracardiac impedance, such as described in Ni et al. U.S. Pat. No. 7,440,803 entitled CLOSED LOOP IMPEDANCE-BASED CARDIAC RESYNCHRONIZATION THERAPY SYSTEMS, DEVICES, AND METHODS, which is incorporated herein by reference in its entirety, including its description of using an implantable device to determine cardiac wall motion. Such information can be stored or used to generate an alert or other indication, such as can be communicated to a user or automated process. In an example, MRI visualization information such as cardiac wall motion can be detected, such as using an MRI or fMRI scanner. Such MRI-visualization-derived cardiac wall motion information can be used to augment, validate, calibrate, or otherwise modify the use of the implantably-obtained cardiac wall motion information or a physiological status (e.g., heart failure status etc.) that can be derived therefrom.

In a calibration example, baseline MRI visualization information such as cardiac wall motion can be detected, such as using an MRI or fMRI scanner. Then, a cardiac stress condition can be induced in the subject, such as by administering a drug (e.g., dobutamine or adenosine) or performing a physical activity stress test. During both the baseline and stress conditions, the implantable cardiac function management device 102 can be used, such as to measure one or more physiological parameters (e.g., impedance-derived cardiac wall motion), or optionally to adjust one or more cardiac function management therapy parameters. A correlation can be computed between a baseline-to-stress change in the MRI visualization information (e.g., cardiac wall motion) and a corresponding baseline-to-stress change in the physiological information measured by the implantable cardiac function management device 102 (e.g., cardiac wall motion). Such correlation can be used to calibrate a later change in the physiological information (e.g., cardiac wall motion) measured by the implantable cardiac function management device 102. For example, this correlation can permit the later change in the physiological information (e.g., cardiac wall motion, such as measured by a change of impedance in Ohms/second) measured by the implantable cardiac function management device 102 to be expressed in terms of the units of the MRI visualization information (e.g., cardiac wall motion, such as measured in mm/second). To create the correlation, a single stress state relative to a baseline can be used, or multiple (e.g., different) stress states can be used, such as to create a correlation look-up table or function. If a good enough correlation exists between the physiological information measured by the implantable cardiac function management device 102 and the MRI visualization information, such correlation can even be used to create an implantably (e.g., chronic ambulatory) obtainable surrogate for the non-implantable, non-ambulatory MRI visualization measurement. Although the above example has described the correlation with an emphasis on calibrating an implantable measurement using MRI visualization information, calibration of the MRI visualization information using the implantably-obtained information can also be performed. In the above example, of particular interest is the synchrony of left ventricular wall motion since it is a measure of left ventricular disease and the effectiveness of cardiac resynchronization therapy. However, septal wall motion, valve motion, or other MRI visualization-based indications can also be of interest.

Although the above example has emphasized a transcardiac or intracardiac impedance to implantably determine cardiac wall motion, the present subject matter can also be applied to an example in which an intracardiac accelerometer (e.g., on an intravascular intracardiac lead) can be used to measure information about cardiac wall motion, such as described in Yu et al. U.S. Patent Application Publication No. US 2007/0129781 A1, entitled CARDIAC RESYNCHRONIZATION SYSTEM EMPLOYING MECHANICAL MEASUREMENT OF CARDIAC WALLS, which is incorporated herein by reference in its entirety, including its description of using an intracardiac accelerometer to measure information about cardiac wall motion.

Also, the above example has emphasized correlating MRI cardiac visualization information of cardiac wall motion to cardiac wall motion obtained from the implantable cardiac function management device 102, such as for use in calibrating a diagnostic parameter of the implantable cardiac function management device 102. However, the techniques described above can also be similarly applied with respect to a cardiac stroke volume, which can also be measured using cardiac imaging visualization information from an MRI or fMRI scanner, and which can also be measured using a transcardiac or intracardiac impedance, such as described in Salo et al. U.S. Pat. No. 4,686,987 entitled BIOMEDICAL METHOD AND APPARATUS FOR CONTROLLING THE ADMINISTRATION OF THERAPY TO A PATIENT IN RESPONSE TO CHANGES IN PHYSIOLOGIC DEMAND, which is incorporated herein by reference in its entirety, including its description of determining cardiac stroke volume using an implantable medical device.

Similarly, although the above example has emphasized correlating MRI cardiac visualization information of cardiac wall motion to cardiac wall motion obtained from the implantable cardiac function management device 102, such as for use in calibrating a diagnostic parameter of the implantable cardiac function management device 102, the techniques described above can also be similarly applied with respect to a respiratory tidal volume, which can also be measured using cardiac imaging visualization information from an MRI or fMRI scanner, and which can also be measured using a transthoracic impedance, such as described in Hauck et al. U.S. Pat. No. 5,036,849 entitled VARIABLE RATE CARDIAC PACER, which is incorporated herein by reference in its entirety, including its description of determining respiratory tidal volume using an implantable medical device. In such an example, the non-baseline condition or conditions need not be drug-induced or exercise-induced, but can instead be obtained by asking the subject to breathe normally (baseline), and breathe deeply (e.g., a non-baseline condition corresponding to the “stress” condition described above).

### Non-MRI Information Examples

Information from one or more non-MRI devices can be used in combination with MRI information (e.g., fMRI functional physiological imaging information or MRI cardiac or pulmonary imaging visualization information), such as to provide one or more indications about cardiac or pulmonary or other cardiovascular function. In an example, an ultrasound apparatus, such as the ultrasound imaging portion 220, can be used to obtain echocardiogram information about the heart 204. For example, echocardiography can use ultrasound and Doppler technology such as to provide imaging information about the heart 204 (e.g., two dimensional or three dimensional images of the heart), information about blood flow (e.g., velocity of blood flow), or information about motion of one or more portions of a heart 204 (e.g., valve opening or closure, cardiac wall motion, septal wall motion, etc.) Echocardiography can provide nearly real-time information about one or more of the heart 204, blood flow, or surrounding tissues. For example, an image of one or more of the heart 204, blood flow, or surrounding tissues can be displayed, such as for viewing by a doctor, nurse, technician, or the like. In such an example, a viewer can be presented with a nearly real-time display of the movement of the tissue of the heart 204 and blood flow (e.g., valve opening or closure, contractions of the heart, blood flow, etc.).

In an example, a positron emission tomography (PET) scanner, such as the PET scanner 216 can be used to obtain imaging information that can provide indications about cardiac function of the heart 204. For example, PET can be used to obtain information that can provide indications of cardiac metabolic activity of the heart 204. PET can involve the introduction of a biologically active radioactive tracer molecule into the body of the patient 202. Information about the rate of uptake of the tracer molecule in the heart 204 and surrounding tissue can provide information about the metabolic activity of the heart 204. For example, an increased rate of uptake of the tracer molecule in the heart 204 can indicate an increase in the metabolic activity of the heart 204.

In an example, a computed tomography (CT) scanner, such as the CT scanner 222, can be used to obtain imaging information about one or more of the heart 204 or surrounding tissues (e.g., coronary arteries etc.) CT can involve the use of multiple X-ray images taken at known angles around a single axis of rotation. For example, multiple two-dimensional images can be taken of a region of the body, such as the heart 204 and surrounding tissue, and the two-dimensional images can be reconstructed into a three-dimensional image. The use of CT can provide advantages as compared to other imaging techniques, such as the ability to obtain higher resolution images at a higher rate of capture. For example, an image can be taken of the heart 204, using a CT scanner, within the timeframe of one contraction of the heart 204. In an example, multiple images of the heart 204 can be taken using a CT scanner over known increments of time. In such an example, information can be obtained that can be used such as to provide indications about one or more of an amount of motion (e.g., displacement, rate, acceleration, etc.), diastolic activity, or systolic activity of one or more portions of the heart 204 (e.g., cardiac walls, septal wall, heart valves, etc.) Such indications can be determined automatically, such as by capturing and recording the CT imaging data and using automated image processing software to provide the indications. In an example, such indications can be determined such as by one or more of a doctor, a nurse, a technician, or the like.

In an example, an X-ray imaging apparatus, such as the X-ray imaging portion 214, can be used to obtain imaging information about one or more of the heart 204 or surrounding tissues. X-ray imaging information can be used, for example, such as to provide structural information about the heart 204 and surrounding tissue. For example, X-ray imaging information can be used to provide an indication of enlargement of one or more portions of the heart 204. An enlargement of one or more portions of the heart 204 can indicate congestive heart failure.

### Combining Information

In an example, information from one or more devices, such as the MRI scanner 218 (e.g., fMRI functional cardiac imaging information and MRI cardiac imaging information), the PET scanner 216 (e.g., cardiac metabolic activity information), the ultrasound imaging portion 220 (e.g., echocardiogram information), the CT scanner 222, and the X-ray imaging portion 214, can be combined. In such an example, the information from one or more of the devices can be used alone or in combination, such as to optimize or otherwise determine a device parameter of an implantable or other ambulatory device, such as the implantable cardiac function management device 102. In an example, imaging information from one or more devices can be combined for concurrent viewing of the information, such as by displaying the information concurrently on a display device. In an example, the imaging information from one or more devices can be recorded, such as by the local external interface device 104, the remote external interface device 106, or the imaging computer 210, such as for review or processing of the information.

In an example, data from an echocardiogram can be used in combination with fMRI cardiac functional imaging information. For example, the echocardiogram imaging information can be used to provide indications of one or more of the amounts of blood flow or the movement of one or more portions of the heart 204 (e.g., cardiac wall movement, septal wall movement, valve opening or closure, etc.), and the fMRI cardiac functional imaging information can be used to provide indications about oxygenation levels of one or more of the blood, the heart 204, or surrounding tissue.

In an example, echocardiogram imaging information can be combined with PET imaging information. The echocardiogram imaging information can be used to provide indications about one or more of the amounts of blood flow or the movement of one or more portions of the heart 204, and the PET imaging information can be used to provide indications about cardiac metabolic activity.

In an example, echocardiogram imaging information can be combined with CT imaging information. The echocardiogram imaging information can be used to provide near real-time indications about one or more of the amounts of blood flow or the movement of one or more portions of the heart 204, and the CT imaging information can be used to provide higher resolution imaging information.

In an example, CT imaging information can be combined with PET imaging information. For example, the CT imaging information can be used to provide indications of one or more of an amount of motion, diastolic activity, or systolic activity of one or more portions of the heart 204, and the PET imaging information can be used to provide indications of cardiac metabolic activity of the heart 204.

In an example, CT imaging information can be combined with fMRI cardiac functional imaging information. For example, the CT imaging information can be used to provide indications of one or more of an amount of motion, diastolic activity, or systolic activity of one or more portions of the heart 204, and the fMRI cardiac functional imaging information can be used to provide indications of one or more of amounts of blood flow or oxygenation levels of the blood, heart 204, or surrounding tissue.

In an example, MRI cardiac imaging information can be combined with PET imaging information. For example, the MRI cardiac imaging information can be used to provide indications of one or more of the amount of motion, diastolic activity, or systolic activity of one or more portions of the heart 204, and the PET imaging information can be used to provide indications of cardiac metabolic activity of the heart 204.

In an example, MRI cardiac imaging information can be combined with MRI cardiac functional imaging information. For example, the MRI cardiac imaging information can be used to provide indications of one or more of the amount of motion, diastolic activity, or systolic activity of one or more portions of the heart 204, and the fMRI cardiac functional imaging information can be used to provide indications of one or more of oxygenation levels of the blood, heart 204, or surrounding tissue, and amounts of blood flow.

In an example, the combined information can be used, such as to select one or more device parameters, such as one or more diagnostic parameters, one or more therapy control parameters, or one or more other device operational parameters of an implantable or other ambulatory device, such as the implantable cardiac function management device 102. In an example, one or more of the multiple indications obtained from the imaging information can be weighted and combined, such as to provide a weighted composite index of heart function. The one or more device parameters can be selected such as to generally increase or maximize the weighted composite index.

### ADDITIONAL NOTES

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile tangible computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

The claimed invention is:

1. An apparatus comprising:
a processor circuit, configured to provide at least one cardiac function management device parameter, of an ambulatory medical device, having a value established at least in part using cardiac functional imaging information obtained from a functional magnetic resonance imaging (fMRI) device.

2. The apparatus of claim 1, comprising:
a port, configured to be communicatively coupled to the fMRI device and to the processor circuit;
wherein the processor circuit is configured to establish the value of the at least one cardiac function management device parameter at least in part using the cardiac functional imaging information obtained from the fMRI device; and
wherein the cardiac functional imaging information obtained from the fMRI device is obtained using the port.

3. The apparatus of claim 1, comprising:
a therapy circuit, configured to be communicatively coupled to the processor circuit and to provide a therapy to a subject;
wherein the cardiac function management device parameter comprises a cardiac function management therapy control parameter configured to control at least one of a therapy timing, a therapy energy level, or a therapy location; and
wherein the processor circuit is configured to control operation of the therapy circuit using the at least one cardiac function management therapy control parameter.

4. The apparatus of claim 1, wherein the at least one cardiac function management device parameter has a value established using information obtained from an imaging apparatus including at least one of an echocardiogram, a computed tomography (CT) scan, a positron emission tomography (PET) scan, magnetic resonance imaging (MRI) scan, or an X-ray image.

5. The apparatus of claim 1, wherein the processor circuit comprises a mode configured to:
establish operative functionality of the ambulatory medical device that is compatible for use during an MRI procedure; and
allow cardiac signal sensing during the MRI procedure.

6. The apparatus of claim 1, wherein the processor circuit comprises a mode configured to vary a value of the at least one cardiac function management device parameter when a subject associated with the ambulatory medical device is undergoing an imaging procedure.

7. The apparatus of claim 1, wherein the at least one cardiac function management device parameter has a value established using cardiac functional imaging information obtained from the fMRI device including information about at least one of a deoxyhemoglobin level or an amount of blood flow.

8. The apparatus of claim 7, wherein the establishing the value of the at least one cardiac function management device parameter comprises establishing the at least one cardiac function management device parameter associated with at least one of a decreased deoxyhemoglobin level indicated by the cardiac functional imaging information obtained from the fMRI device or an increased amount of blood flow indicated by the cardiac functional imaging information obtained from the fMRI device.

9. The apparatus of claim 7, wherein the establishing the value of the at least one cardiac function management device parameter comprises:
establishing an efficiency indicator value of the heart using at least one of the information about the deoxyhemoglobin level or amount of blood flow obtained from the fMRI device; and
establishing the at least one cardiac function management device parameter associated with an increased efficiency indicator value.

10. The apparatus of claim 1, wherein the at least one cardiac function management device parameter of the ambulatory medical device is configured to provide a correlation between a measurement of the ambulatory medical device to a functional physiological measurement obtained using an imaging device.

11. A device-readable medium including instructions that, when performed by the device, comprise:
establishing a value of at least one cardiac function management device parameter, of an ambulatory medical device, at least in part using cardiac functional imaging information obtained from a fMRI device.

12. The device-readable medium of claim 11, comprising instructions to provide a therapy to a subject using the at least one cardiac function management device parameter, and wherein the at least one cardiac function management device parameter comprises a cardiac function management therapy control parameter configured to control at least one of a therapy timing, a therapy energy level, or a therapy location.

13. The device-readable medium of claim 11, wherein the establishing the value of the at least one cardiac function management device parameter comprises using information obtained from an imaging apparatus including at least one of an echocardiogram, a computed tomography (CT) scan, a positron emission tomography (PET) scan, an MRI scan, or an X-ray image.

14. The device-readable medium of claim 11, comprising instructions to establish operative functionality of the ambulatory medical device that is compatible for use during an MRI procedure and that allows cardiac signal sensing during the MRI procedure.

15. The device-readable medium of claim 11, comprising instructions to vary the value of the at least one cardiac function management device parameter when a subject associated with the ambulatory medical device is undergoing an imaging procedure.

16. The device-readable medium of claim 11, wherein the establishing the value of the at least one cardiac function management device parameter using the cardiac functional imaging information obtained from the fMRI device comprises using information about at least one of a deoxyhemoglobin level or an amount of blood flow.

17. The device-readable medium of claim 16, wherein the establishing the value of the at least one cardiac function management device parameter comprises establishing the value of the at least one cardiac function management device parameter associated with at least one of a decreased deoxyhemoglobin level indicated by the cardiac functional imaging information obtained from the fMRI device or an increased amount of blood flood flow indicated by the cardiac functional imaging information obtained from the fMRI device.

18. The device-readable medium of claim 16, wherein the establishing the value of the at least one cardiac function management device parameter comprises:
establishing an efficiency indicator value of the heart using at least one of the information about the deoxyhemoglobin level or amount of blood flow obtained from the fMRI device; and
establishing the at least one cardiac function management device parameter associated with an increased efficiency indicator value.

19. The device readable medium of claim 11, wherein the at least one cardiac function management device parameter of the ambulatory medical device is configured to provide a correlation between a measurement of the ambulatory medical device to a functional physiological measurement obtained using an imaging device.

20. A method comprising:
establishing, using a processor circuit, a value of at least one of a cardiac function management device parameter, of an ambulatory medical device, at least in part using cardiac functional imaging information obtained from a fMRI device.

21. The method of claim 20, wherein the establishing the value of the at least one cardiac function management device parameter using cardiac functional imaging information obtained from the fMRI device comprises using information about at least one of a deoxyhemoglobin level or an amount of blood flow.

22. The method of claim 20, wherein the establishing the value of the at least one cardiac function management device parameter comprises establishing the value of the at least one cardiac function management device parameter associated with at least one of a decreased deoxyhemoglobin level indicated by the cardiac functional imaging information obtained from the fMRI device or an increased amount of blood flood flow indicated by the cardiac functional imaging information obtained from the fMRI device.

23. The method of claim 20, comprising establishing the at least one cardiac function management device parameter of the ambulatory medical device to provide a correlation between a measurement of the ambulatory medical device to a functional physiological measurement obtained using an imaging device.
